(19) United States
(12) Patent Application Publication
(10) Pub. No.: US 2026/0207035 A1
(43) Pub. Date: Jul. 23, 2026

(54) Control apparatus for an endoscopic apparatus, handpiece for a flexible endoscopic apparatus and endoscopic apparatus

(71) Applicant: KARL STORZ SE & Co. KG, Tuttlingen (DE)

(72) Inventor: Alexander Fuchs, Steißlingen (DE)

(73) Assignee: KARL STORZ SE & Co. KG, Tuttlingen (DE)

(21) Appl. No.: 19/141,502

(22) PCT Filed: Dec. 20, 2023
(86) PCT No.: PCT/EP2023/086337

§ 371 Date: Jun. 20, 2025

(30) Foreign Application Priority Data
Dec. 20, 2022 (DE) 102022134137.2

(51) Int. Cl. A61B 1/005; A61B 1/00

(52) U.S. Cl. CPC A61B 1/0052; A61B 1/00103; A61B 1/00105; A61B 1/0057

(57) ABSTRACT

The invention relates to a control apparatus for an endoscopic apparatus with a bendable shaft portion, wherein the control apparatus has a control element, an actuating element and at least one Bowden cable with at least one cable element, wherein the at least one cable element is guided at least partially along the control element and has a clamping device with at least one clamping element, wherein the at least one clamping element is connected to the control element in a non-positive-locking and/or positive-locking manner, and the at least one cable element is fastened to the control element by means of the clamping device. Furthermore, the invention relates to a handpiece and an endoscopic apparatus.

The invention relates to a control apparatus for an endoscopic apparatus with a bendable shaft portion, wherein the control apparatus has a control element, an actuating element and at least one Bowden cable with at least one cable element, wherein the at least one cable element is guided at least partially along the control element, and has a clamping device with at least one clamping element. Furthermore, the invention relates to a handpiece for a flexible endoscopic apparatus and an endoscopic apparatus.

Bendable instruments and endoscopes which can be operated manually and/or robotically are often used in medical and non-medical applications. Reusable endoscopes or instruments often use a plurality of steering wires, which are arranged around pivoting elements of a mechanical joint system on the distal side and fastened to a drivable swash plate on the proximal side. For endoscopes and instruments that are only used once, such a complex design and assembly is not feasible. For disposable endoscopes and instruments, it is known to use a circumferential Bowden cable or a few Bowden cables for the joint mechanics on the distal and proximal sides. In the proximal-side joint mechanism, the pull wire ends of the circumferential Bowden cable or the pull wires of the Bowden cables are usually connected to one another and fixed to a steering wheel.

For example, two pull wire ends are inserted into a crimp sleeve in opposite directions and pressed together, wherein the crimp sleeve is inserted into a groove on the steering wheel. The positive connection between this pull wire arrangement with the crimp sleeve and the steering wheel ensures that when the steering wheel is turned, one pull wire is tightened and the other is slackened. To secure such a pull wire arrangement on the steering wheel against unintentional loosening, it is also known to glue the crimp sleeve in the groove of the steering wheel. The crimp sleeve may also be fastened to the steering wheel by means of a securing clip. These types of fastening are complex, time-consuming and potentially error-prone due to the installation. In addition, such assembly is very complex to implement in an automated process. Another disadvantage is that the securing clip usually has to engage mechanically with the steering wheel, which increases the installation space and makes it difficult to scale the diameter of the steering wheel for different endoscopes.

The object of the invention is to improve upon the prior art.

The object is achieved by a control apparatus for an endoscopic apparatus with a bendable shaft portion, wherein the control apparatus has a control element, an actuating element and at least one Bowden cable with at least one cable element, wherein the at least one cable element is guided at least partially along the control element and has a clamping device with at least one clamping element, wherein the at least one clamping element is connected to the control element in a non-positive-locking and/or positive-locking manner and the at least one cable element is fastened to the control element by means of the clamping device.

Thus, a control apparatus for an endoscopic apparatus is provided in which, due to the design of the clamping device, scalability of the control element for different endoscopic apparatuses is possible. It is particularly advantageous that the non-positive-locking and/or positive-locking connection of the at least one clamping element of the clamping device to the control element can be formed independently of the fastening of the at least one cable element to the control element by means of the clamping device. As a result, the at least one clamping element does not necessarily have to surround the at least one cable element and/or clamp it directly to the control element. This eliminates the need for an eyelet for mechanical engagement between the outer diameter and the center of the control element, allowing a smaller minimum diameter of the control element.

Due to the independent and free designability of the clamping element, which is connected to the control element in a non-positive-locking and/or positive-locking manner, and the fastening of the at least one cable element to the control element by means of the clamping device, the diameter of the control element can be varied. The adaptation of the relevant diameter of the control element for different variants of endoscopic apparatuses is necessary to allow the user to operate the apparatus in a haptically and ergonomically consistent manner. Control elements with different diameters should have similar haptics so that, when the actuating element is actuated, the extent of the bending of the bendable shaft portion is analogous to the rotation of the relevant steering wheel.

In addition, the design of the clamping device and/or the at least one clamping element allows the mounting direction of the clamping device to be determined more flexibly and to be specifically adapted to the mounting directions of the other components of the control apparatus and/or of the proximal-side joint mechanism, so that a simple, fast and error-free mounting of the clamping device in a manual and/or automated process is possible. This allows for control elements with both the largest and smallest possible diameters. By allowing a control element with a smaller, minimal diameter, the pull wires, which are at least partially guided around the control element, are closer together and the articulation is less stiff and therefore more comfortable for the user to handle.

Because the clamping element for connection to the control element does not have to be specifically adapted to the arrangement of the cable elements connected on the proximal side, differently designed control elements can be easily used with the clamping device. Thus, the control apparatus and the handpiece can be quickly and easily adapted to different flexible endoscopes or instruments, e.g., having different shaft diameters, because, depending upon the shaft diameter in each case, one or more Bowden cables with correspondingly adapted cross sections are usually used. To adapt to the properties of the relevant endoscope or instrument, only the control element and optionally the clamping element need to be replaced. This allows the control apparatus to be adapted to the desired haptics in a simple and flexible manner.

An essential idea of the invention is to provide a modularly adaptable control apparatus for a proximal-side joint mechanism of various flexible endoscopes and instruments, in which the non-positive-locking and/or positive-locking connection of the at least one clamping element of the clamping device to different control elements is made possible and at the same time the at least one cable element is fastened to the control element by means of the clamping device in such a way that a mounting direction when assembling the control apparatus can be largely freely selected and/or adapted to the assembly direction of the other components of the control apparatus and/or the handpiece.

The following terminology is explained:

A “control apparatus” is in particular any apparatus which allows the bending of a bendable shaft portion of an endoscopic apparatus by actuation on the proximal side. The control apparatus is in particular a component of the proximal-side joint mechanism or forms the proximal-side joint mechanism. The control apparatus comprises in particular a control element, an actuating element, a shaft-hub connection and at least one Bowden cable with at least one cable element. Furthermore, the control apparatus can have a holding frame. The parts and/or components of the control apparatus comprise, in particular, plastics material and are preferably manufactured as plastics material injection-molded parts.

An “endoscopic apparatus” is, in particular, a medical or industrial apparatus or instrument for endoscopic examination, observation and/or manipulation in a human, animal and/or industrial cavity. An endoscopic apparatus can be an endoscope or an endoscopic instrument.

An “endoscope” is in particular a medical or industrial device for endoscopic examination and inspection of a human or animal body cavity and/or an industrial cavity, such as a tube. The endoscope has in particular a handpiece, a shaft, a light source, a light guide, and a camera. The endoscope is in particular a video endoscope, which has digital image recording and image transmission. A video endoscope is in particular a chip-on-the-tip (COTT) endoscope, wherein the image sensor is designed as a chip, e.g., a CMOS chip, and is arranged on the distal end portion of the shaft of the video endoscope. The image data recorded by the image sensor can in particular be transmitted electronically through the shaft in the proximal direction to the handpiece and further to a display system and/or to an image processing unit in order to display the endoscopic image to the user. In addition to medical and veterinary applications, an endoscope and/or video endoscope may, however, also be used for industrial purposes, for example for visual inspection in hard-to-reach cavities. In industrial applications, an endoscope is often referred to as a borescope.

An “endoscopic instrument” is in particular any mechanical or mechanical-electrical operating unit which can be used in particular for manipulating human or animal tissue. The endoscopic instrument has in particular a handpiece, a flexible shaft, and a tool and/or an optical system for observing a viewing area. A flexible endoscopic instrument usually does not have its own optics for taking an endoscopic image, but can be used in particular together with a flexible endoscope. The endoscopic instrument may in particular have a gripping tool, a cutting tool, a needle holder, a clip placement device, and/or another type of tool.

A “flexible endoscope” or a “flexible endoscopic instrument” is understood in particular to mean that the endoscope or the endoscopic instrument has a flexible shaft. A “flexible shaft” is in particular a partially or fully flexible elongated tube. The flexible shaft is in particular designed to be inserted into a cavity to be examined endoscopically-for example, a body cavity or a technical opening in industrial applications. Typically, the shaft has an outer diameter in a range of 4 mm to 10 mm. The flexible shaft has, in particular, at least one operating channel on the inside for flushing or for passing operating instruments through. The flexible shaft has at least one bendable shaft portion. The “bendable shaft portion” refers in particular to a bendable distal end portion of the shaft. In order to bend the shaft portion, the flexible shaft has, in particular on the inside, a distal-side mechanical joint system with joint members arranged side-by-side in a row in the longitudinal direction, and at least one circumferential Bowden cable guided around the outside of the joint members on both sides. The housing of the Bowden cable can be fastened, on the distal side, in front of the first joint member or, in the distal end portion, in front of the camera head-for example, by gluing. A flexible endoscope is, in particular, a sterile, disposable endoscope for single use. The flexible endoscope can be, for example, a bronchoscope or an incubation endoscope.

A “handpiece” is, in particular, a handheld part and/or retaining part for an endoscopic apparatus, an endoscope and/or instrument. In particular, the handpiece can be a handle for manual and/or automatic operation. In particular, the handpiece has a housing. For example, the housing can be made up of two lateral handle shells that are connected to one another. At the distal end of the handpiece, the handpiece can be connected, e.g., by means of an adapter, to the proximal end of a shaft of the endoscope or instrument. The housing of the handpiece is made of plastics material in particular and is preferably manufactured as a plastics material injection-molded part.

“Distal-side” and “distal” mean an arrangement and/or a corresponding end or portion that is close to the patient's body and therefore remote from the user. Accordingly, “proximal side” and “proximal” are understood to mean an arrangement and a corresponding end or portion close to the user and thus remote from the patient's body. Thus, the ‘proximal direction’ is a direction towards the proximal end of the handpiece and/or the endoscope or endoscopic instrument. Accordingly, the “distal direction” is the opposite direction directed toward the tip of the bendable shaft portion.

A “control element” is in particular an element which causes the cable element of the at least one Bowden cable to move, and thus the bendable shaft portion to bend. A control element can, for example, be an idler pulley or a steering wheel. The control element does not necessarily have to be circular in shape, but can also be oval, drop-shaped or shaped in another way.

An “actuating element” is in particular an element whose movement actuates the control element. An actuating element can, for example, be an articulation lever with a handle at the end, wherein the lever itself is guided through the housing of the handpiece and connected to the control element.

A “Bowden cable” is in particular a movable machine element for transmitting a mechanical movement. A Bowden cable has in particular a housing (also called an outer cable) that is flexible but pressure-resistant in the pulling direction, and a cable element arranged inside the housing (also called an inner cable, pull cable, or pull wire). The cable element is movable within the housing, in particular in the longitudinal direction of the Bowden cable. The cable element is in particular a steel wire or wire cable. By moving the cable element by means of the control element, the cable element is displaced relative to the housing of the Bowden cable in the longitudinal direction of the Bowden cable, whereby the bendable shaft portion can be bent by a set amount in a set direction by means of the distal mechanical joint system. The housing of the Bowden cable is, at least in part, substantially cylindrical. The Bowden cable can, for example, have an outer diameter in the range of 0.10 mm to 3.00 mm, in particular 0.30 mm to 1.00 mm, preferably 0.40 mm to 0.75 mm.

A “holding frame” (also called a “bearing block”) is in particular a supporting structure inside the housing. The support frame is connected in particular to the housing and/or a handle shell on the inside. The support frame is in particular designed having two opposite support arms, between which the control element is arranged. The control element is connected to the two support arms of the support frame, in particular by means of a shaft.

A “clamping device” is in particular a three-dimensionally shaped component which has at least one clamping element by means of which the clamping device can be connected to a control element in a non-positive-locking and/or positive-locking manner, wherein the clamping device is simultaneously designed such that at least one cable element can be fastened to the control element. The clamping device is in particular detachably connected to the control element. By means of the clamping device and/or the at least one clamping element, in particular a flat portion and/or flat portions on opposite sides of the control element are clamped on and/or clamped in and thus spatially surround the control element. The clamping device has in particular plastics material, such as polypropylene. The clamping device is preferably designed in one piece, but can also be in two or more parts. For example, the clamping device can also have two clamping clips. Likewise, the clamping device may additionally have a securing clip to secure its spatial position on the control element.

A “clamping element” is in particular an elastic element which is or can be connected directly to the control element in a non-positive and/or positive manner. The clamping element is in particular a component of the clamping device and/or designed as a portion of the clamping device. The clamping element can be, for example, a spring clamp or a locking hook with a locking lug.

A “non-positive connection” is in particular a connection in which a normal force is applied to the surfaces to be interconnected. This prevents the surfaces from moving relative to one another due to static friction. In the non-positive connection, the holding force of the clamping element is used to fix and/or fasten the clamping device, the at least one cable element and/or the pull wire arrangement to the control element. The non-positive connection is in particular a clamping connection. The non-positive connection is in particular reversible and releasable, in particular by loosening the clamping element.

A “positive connection” is in particular a connection in which at least two connection partners engage with one another. In the case of a positive connection, a blocking occurs in particular when one connection partner is in the way of the other connection partner, so that the connection partners or if the power transmission is interrupted do not come loose. The positive connection between two connection partners can also be realized by means of a third connecting element, for example a pin or bolt. The positive connection may, for example, be a detachable connection by means of a tongue and groove, a key or interlocking gears. The two connection partners are in particular the at least one clamping element and the control element. However, the clamping element may also simultaneously form a non-positive and positive connection with the control element.

In a further embodiment, the control apparatus has at least one second Bowden cable and optionally further Bowden cables, each with at least one cable element.

Thus, instead of a circumferential Bowden cable, two or more Bowden cables can also be guided in the control apparatus by means of the control element. In the case of two Bowden cables, two cable elements working against one another can be guided on opposite sides of the control element. These two cable elements can also be connected to one another on the proximal side and thus be designed as a continuous cable element.

A “second Bowden cable” and “further Bowden cables” correspond in their design and function to the Bowden cable defined above. However, these can also have other properties, such as different diameters, and be guided differently and/or at different distances from one another in the control apparatus, the handpiece and/or through the flexible shaft to the bendable shaft portion.

In order to securely connect the at least one clamping element to the control element, the control element can have at least one counter-clamping portion for engaging and/or enclosing by means of the at least one clamping element.

In a further embodiment of the control apparatus, the counter-clamping portion is designed as a recess, an undercut and/or a shaped surface in and/or on the control element.

Thus, the counter-clamping portion has a specifically shaped region or defined point in, on and/or around which the at least one clamping element or the clamping elements can apply their clamping force by engaging and/or enclosing.

A “recess” is in particular an incision and/or an opening. The recess may, for example, be a notch, a groove, and/or another type of incision in the surface of the support frame.

An “undercut” is in particular a construction element which protrudes freely from the control element. An undercut can be, for example, a rib.

A “shaped surface” is in particular a portion of a surface of the control element as a specially shaped counter-clamping surface or portion. The shaped surface can change evenly or in steps, particularly in the mounting direction. For example, the shaped surface may have a rounded or curved shape or may widen conically and/or step-like in the mounting direction and thus in the sliding direction of the clamping element. The shaped surface may also have one or more corners for gripping and/or one or more incisions for engaging one or more clamping elements.

In order to allow simple operation of the control element and smooth movement of at least one cable element, the control element is designed as a steering wheel with an axis of rotation.

Thus, by operating the actuating element, a rotation of the steering wheel as a control element and a corresponding displacement of the cable elements of the Bowden cables is caused.

As “axis of rotation” is in particular a fixed axis of rotation about which the steering wheel rotates. For this purpose, the steering wheel can have a shaft-hub connection.

In a further embodiment of the control apparatus, the control element and/or the steering wheel is circular, substantially oval or drop-shaped.

Thus, by selecting the relevant shape of the control element and/or steering wheel, the desired haptics when operating the control apparatus can be specifically adjusted and adapted to the available installation space.

Compared to a circular steering wheel with a fixed radius, a substantially oval or drop-shaped design of the control element has the advantage that the lever with which the control element acts remains substantially the same size as a circular steering wheel which has the maximum diameter of the oval or drop-shaped control element as a fixed diameter. This provides identical haptics. Starting from a larger circular steering wheel, in the case of a substantially oval or drop-shaped control element, the diameter of the control element is predominantly reduced and only left at the location where the clamping device and/or the connected cable elements are arranged. While the reduced diameter is substantially partially circular or semi-circular, the location of the control element with the clamping device has a larger diameter relative to the axis of rotation and thus a larger rounding on the circumference of the control element, which merges on both sides into a transition region into the partially or semi-circular shape, so that the control element has an essentially oval or drop shape. The advantage here is that the proximal-side connection location of the cable elements, for example a crimp, is always located in the same location, even with an oval or drop-shaped control element, as with a circular steering wheel having a larger diameter. Thus, the same manufacturing apparatus can be used to crimp the cable elements. Due to the oval or drop shape, the loaded pull wires always rest on the reduced diameter of the control element when the bendable shaft portion is deflected and thus the control apparatus is actuated. Thus, despite the oval or drop shape, a smaller radius is possible over a wide region of the circumference of the control element and a larger radius is only present in the region of the clamping device. This provides a compact, space-saving control element that has the haptics of a larger circular steering wheel.

In order to provide a clamping device that can be easily mounted on the control element, the clamping device can be at least partially ring-shaped.

Thus, the clamping device can be designed as a ring clip which is mounted in the axial direction of the control element and/or steering wheel. Here, the ring clip is fastened to the control element and/or steering wheel by means of a snap mechanism of the clamping element or clamping elements which enclose the control element and/or the steering wheel in the axial direction. At the same time, the proximal-side connection location of the cable elements, for example connected in a crimp sleeve, is held in the groove on the steering wheel from two sides from different spatial directions and is therefore not displaceable. In principle, it should be emphasized that the clamping device can only be partially ring-shaped and thus only partially surrounds the outer circumference of the control element and/or steering wheel. Preferably, the clamping device is fully ring-shaped, so that it surrounds the entire circumference of the control element and/or steering wheel and in particular the circumferential surface with the groove for guiding the cable elements is at least partially or fully surrounded by the ring clip. Due to the design of the clamping device as a ring clip, no eyelet is necessary in the surface of the control element between its circumference and the axis of rotation, as is required with known securing clips which engage in an eyelet. This allows the diameter of the control element and/or steering wheel to be reduced as much as the shaft-hub connection with the articulation lever allows for actuation.

In a further embodiment of the control apparatus, the clamping device is fastened with the at least one clamping element in a fastening direction along the axis of rotation of the steering wheel.

As a result, the mounting direction of the clamping element and/or the clamping device can be realized along the axis of rotation of the control element and/or steering wheel, which usually corresponds to the mounting direction of the other components of the control apparatus, whereby the mounting of the entire control apparatus can be carried out in a simplified, faster and automated manner.

“Along the axis of rotation” is understood in particular to mean that the fastening direction is substantially in the same direction as the longitudinal direction of the axis of rotation. The fastening direction does not have to run exactly along the direction of rotation, but can also be coaxial or parallel to the axial direction of the axis of rotation.

In order to secure the proximal connection of the cable elements from the side from the outside, the clamping device is fastened with the at least one clamping element in a fastening direction substantially orthogonal to the axis of rotation of the steering wheel.

As a result, the clamping device or the clamping element is pressed laterally, particularly from the outside, against the groove with the connected cable elements during fastening and clamps and/or engages accordingly. For this purpose, the clamping device preferably has a groove, which is located, for example, between two clamping elements, in which groove the connection location and/or the crimp sleeve with the connected cable elements is accommodated.

In a further embodiment of the control apparatus, the clamping device has at least two clamping elements, wherein the at least two clamping elements are arranged on opposite sides of the control element and/or steering wheel.

This ensures that the control element and/or the steering wheel are clamped by means of at least two clamping elements.

Preferably, the at least two clamping elements are arranged on the opposite flat sides of the control element or the disc surfaces of the steering wheel, which have a significantly greater extent than the thickness with the lateral groove of the control element or the steering wheel.

In order to guide and control the at least one cable element or the cable elements in a targeted manner, the control element and/or the steering wheel can have at least one groove for guiding the at least one cable element at least partially along the control element and/or the steering wheel.

In a further embodiment, the control apparatus has two cable elements, each with a proximal end, and the proximal ends of the two cable elements are connected to one another by means of a connecting means.

A “connecting means” can be any type of connecting means that creates a tensile connection between the two proximal ends of the two cable elements. A connecting means can be an integral, non-positive-locking and/or positive-locking connecting means. For example, the connecting means is a crimp sleeve into which the two proximal ends of the cable elements are inserted in opposite directions and pressed together.

In order to ensure secure clamping of the connected proximal ends of the cable elements, the connecting means is arranged in the groove of the control element and/or the steering wheel and/or in the clamping device and is fastened and/or secured to the control element and/or the steering wheel by means of the clamping device.

It is particularly advantageous that the connecting means has a larger diameter than the proximal ends of the cable elements, so that the proximal ends connected in the connecting means or by means of the connecting means have a larger diameter at this connection location, which in turn is useful for clamping between the clamping device and the groove of the control element and/or of the steering wheel.

In a further embodiment, the ring-shaped clamping device is arranged externally around the groove of the control element and/or the steering wheel and/or around the at least one cable element.

As a result, during its guiding, the cable element within the groove is limited on the outside by an inner wall of the ring-shaped clamping device. In the event of a fault, this prevents a loose cable element from being thrown outward when it breaks off and from causing damage inside the housing.

In order to guide the cable elements coming from the distal joint mechanism in a targeted manner along the circumference and/or in the groove of the control element and/or the steering wheel, the clamping device has at least one guide element for guiding the at least one cable element.

The guide element can, for example, be a rail open on one side or a pipe portion which is arranged on the clamping device along the outer diameter of the control element and/or of the steering wheel.

In a further embodiment of the control apparatus, the clamping device has at least one anti-rotation element for preventing rotation of the clamping device on the control element and/or the steering wheel.

This prevents rotation of the ring clip about the axis of rotation and thus the circumference of the control element and/or steering wheel, particularly when the clamping device is designed as a ring clip. The anti-rotation element can, for example, be a tab on the clamping device which engages with the control element and/or the steering wheel and thereby fixes the clamping device in a rotationally secure manner. The anti-rotation element can also be designed as a separate securing clip, which presses against the clamping device and fixes it spatially.

In order to ensure that the ring clip is securely seated on the control element and/or steering wheel, it is guided and/or held in position along the axis of rotation of the control element and/or steering wheel, in particular at a plurality of points on the diameter. The guidance when fitting the ring clip can also be ensured by two or more anti-rotation elements, before they ensure the precise position of the ring clip on the steering wheel.

In a further aspect of the invention, the object is achieved by a handpiece for a flexible endoscopic apparatus with a bendable shaft portion, wherein the handpiece has a control apparatus as described above.

Thus, a handpiece with a control apparatus is provided, wherein the control apparatus can be flexibly adapted depending on the type of flexible endoscopic apparatus and the desired haptics during articulation. This also allows the handpiece to be used for different flexible endoscopic apparatuses, while the control apparatus can be quickly and easily adapted and/or modified depending on the requirements of the endoscopic apparatus due to its modular design. Due to the designability of the control element and the clamping device, the available installation space in the housing of the handpiece can be optimally used and, even with a large lifting length of the control element, the available installation space is not significantly restricted by the control element itself.

In an additional aspect of the invention, the object is achieved by an endoscopic apparatus, in particular a flexible endoscope or a flexible endoscopic instrument, having a shaft and a bendable shaft portion, wherein the bendable shaft portion can be bent by means of at least one Bowden cable, and having a handpiece arranged at a proximal end of the shaft, wherein the endoscopic apparatus has a control apparatus as described above or a handpiece as described above.

Thus, a flexible endoscope or a flexible endoscopic instrument having a handpiece that is inexpensive to manufacture and easy to assemble is provided for single use, such that re-use and the otherwise necessary autoclaving can be dispensed with. Above all, a largely identical handpiece, in which only the control element and/or the clamping device can have different designs, provides an easy-to-use endoscope or endoscopic instrument, especially for the user, which does not require the user to adjust to the change between different types of endoscopes or instruments when operating the handpiece.

The invention will be explained in more detail below with reference to an exemplary embodiment. In the figures:

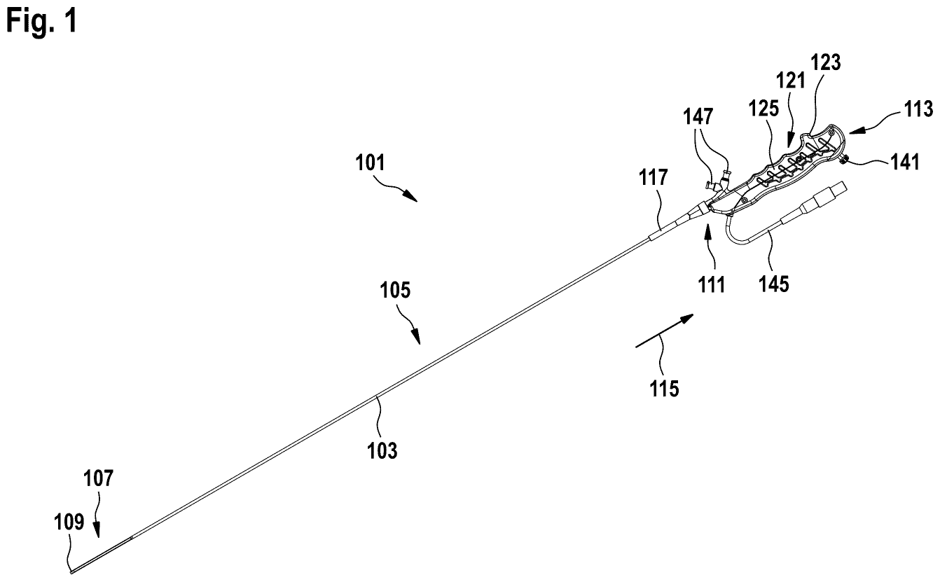

FIG. 1 is a three-dimensional view of an endoscope having a flexible shaft and a handpiece,

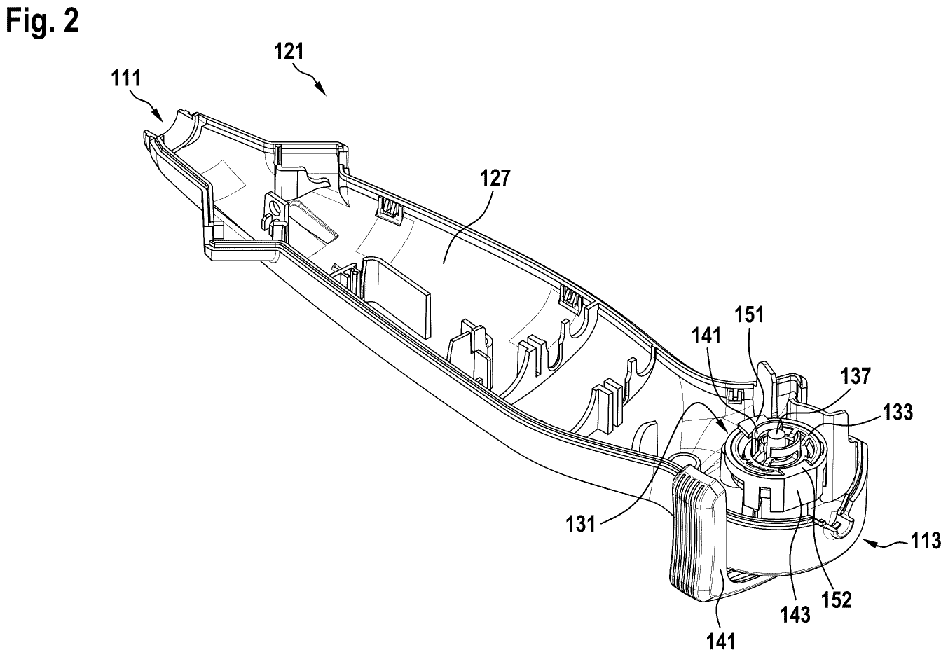

FIG. 2 is a three-dimensional representation of the opened handpiece with a control apparatus having a ring clip on a steering wheel,

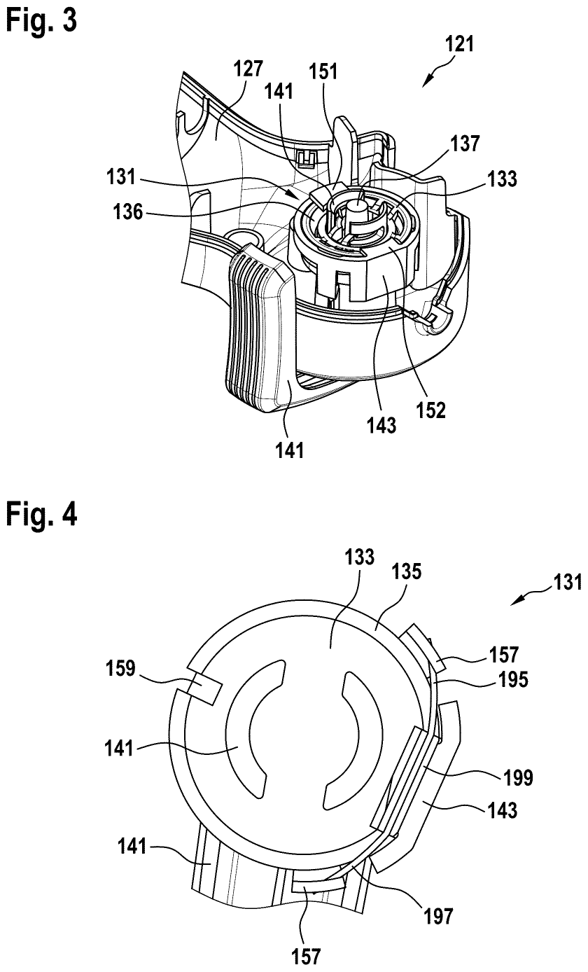

FIG. 3 is a three-dimensional view of an enlargement of a control apparatus with the ring clip on the steering wheel of FIG. 2,

FIG. 4 is a cross-sectional view of the steering wheel with the ring clip from FIG. 3 with a cutting plane in the region of the pull wires,

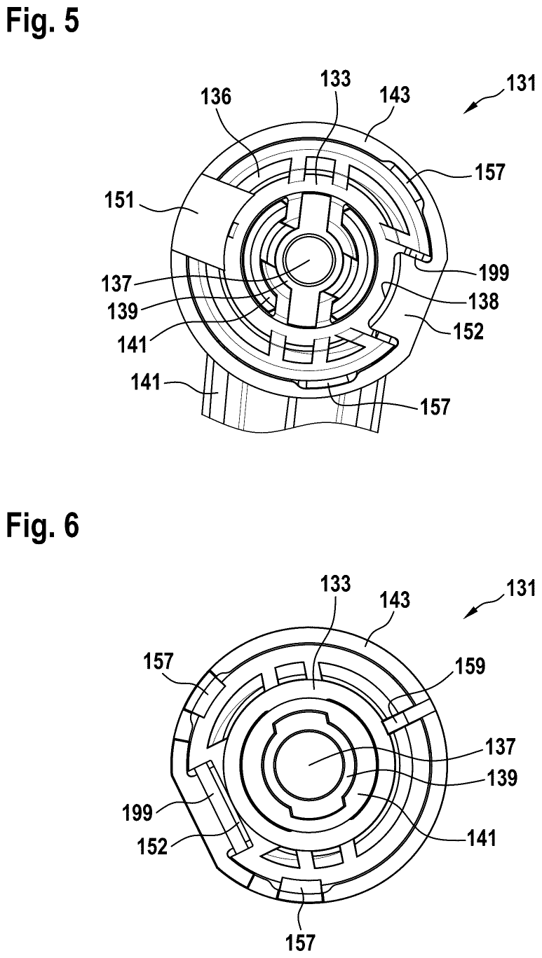

FIG. 5 is a plan view of the steering wheel with the ring clip from above,

FIG. 6 is a plan view of the steering wheel with the ring clip from below,

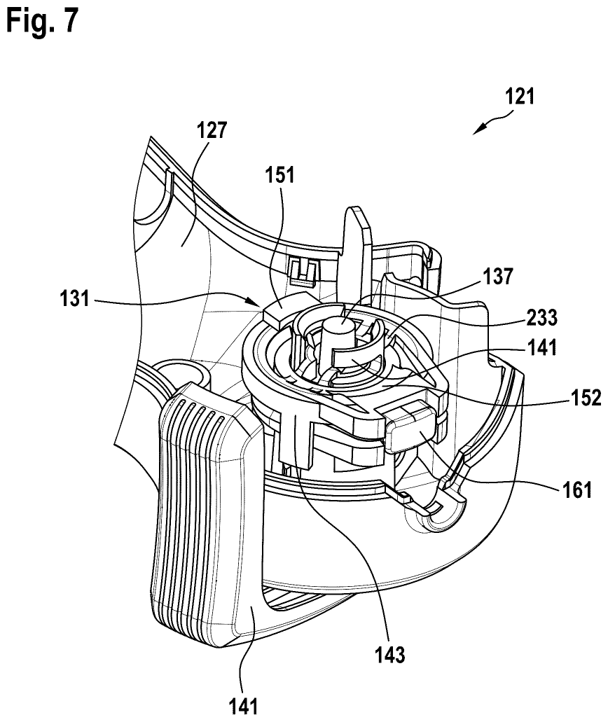

FIG. 7 shows an alternative control apparatus with a ring clip on a drop-shaped steering wheel with an additional securing clip,

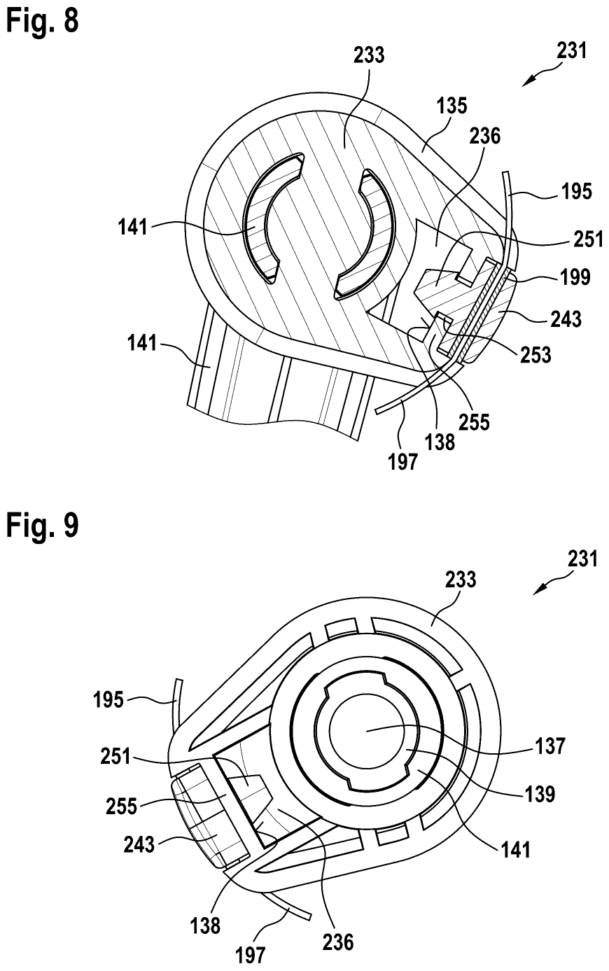

FIG. 8 shows an alternative control apparatus with a drop-shaped steering wheel and a retaining clip in an opening, and

FIG. 9 is a plan view of the drop-shaped steering wheel with the securing clip from FIG. 8 from below.

An endoscope 101 has a shaft 103 with a flexible shaft portion 105 and a distal bendable shaft portion 107. A camera head 109 is arranged in a distal end portion of the bendable shaft portion 107. A proximal end of the shaft 103 is connected to a distal end 111 of a handpiece 121 by means of an adapter 117. The handpiece 121 has a housing 123, which is formed by an upper handle shell 125 and a lower handle shell 127. In a proximal portion in a proximal direction 115 in front of a proximal end 113 of the handpiece 121, the handpiece 121 has an articulation lever 141 which is guided inward into the housing 123. In a distal portion in front of the distal end 111, the handpiece 121 has two valve bodies 147 and a power cable 145 (FIG. 1).

The handpiece 121 (FIG. 2 shows a view of the interior of the lower handle shell 127 of the handpiece 121 with the upper handle shell 125 removed) has a control apparatus 131 on the inside. Other components typically accommodated in the handpiece 121, such as a printed circuit board and a bearing block with an upper bearing arm and a lower bearing arm for supporting a drive shaft 137 of the control apparatus 131, are not shown in FIG. 2. A circular steering wheel 133 is fastened to the drive shaft 137 of the control apparatus 131. The steering wheel 133 has a circumferential guide groove 135 on its side face along its circumference. The steering wheel 133 is fully surrounded by a ring clip 143, which has a first clamping element 151 and a second clamping element 152. The articulation lever 141 for actuating the control apparatus 131 is connected to the steering wheel 133 via the drive shaft 137 and a hub 139.

The ring clip 143 has been mounted in the direction of the drive shaft 137 and engages with its first clamping element 151 in a recess 136, while the second clamping element 152 engages with a clamping surface 138. In the region of the second clamping element 152, the proximal-side ends of a first pull wire 195 and a second pull wire 197 are accommodated in a crimp sleeve 199 and pressed together, so that the two pull wires 195, 197 are connected in a high-tension manner. The crimp sleeve 199 with the connected first pull wire 195 and the second pull wire 197 is accommodated in the guide groove 135 and securely clamped in position by means of the inner wall of the ring clip 143 (see FIG. 4 to 6). On both sides of the crimp sleeve 199, the ring clip 143 has a guide element 157 for guiding the first pull wire 195 and the second pull wire 197 (in FIG. 4, a further course of the first pull wire 195 and the second pull wire 197 along the circumference of the steering wheel 133 in the direction of a distal joint mechanism is not shown). Furthermore, the ring clip 143 has a tab-shaped anti-rotation element 159 (see FIG. 6), which prevents rotation of the ring clip 143 about the circumference of the steering wheel 133.

Thus, a control apparatus 131 is provided with a circular steering wheel 133 and a ring clip 143, wherein the ring clip 143 can be easily mounted in the direction of the drive shaft 137 and ensures a secure holding of the crimp sleeve 199 with the connected first pull wire 195 and the second pull wire 197 in the guide groove 135 by means of the ring clip 143.

A user actuates the articulation lever 141 to bend the bendable shaft portion 107 of the endoscope 101. Due to the arrangement of the steering wheel 133 on the rotatable drive shaft 137 for conjoint rotation therewith, which drive shaft is connected to the articulation lever 141, the steering wheel 133 is rotated by pivoting the articulation lever 141, and as a result the pull wires 195, 197 are displaced accordingly along the guide groove 135. As a result, the bending of the bendable shaft portion 107 is manually controlled by the user of the endoscope 101.

In an alternative of the control apparatus 131 shown in FIG. 7, it has a drop-shaped steering wheel 233. The ring clip 143 is arranged around the round portion of the drop-shaped steering wheel 233 as described above, externally surrounding it, and is inserted internally into the tapered drop-shaped part. The crimp sleeve 199 connecting the two pull wires 195, 197 is held in the guide groove 135 by the ring clip 143 as described above. In addition, the control apparatus 131 has a securing clip 161, which presses from the outside through a hole in the side wall in the tapered part of the drop-shaped steering wheel 233 against the inner outer wall of the ring clip 143 and thus secures the ring clip 143 in its position. Otherwise, the control apparatus 131 is designed as described above and is operated as described above.

An alternative control apparatus 231 has a drop-shaped steering wheel 233 and an articulation lever 141. In the tapered part of the drop-shaped steering wheel 233, an opening 236 is arranged between the drive shaft 137 and the guide groove 135 through the thickness of the drop-shaped steering wheel 233. The ends of the first pull wire 195 and the second pull wire 197 are in turn fastened together in a crimp sleeve 199. The crimp sleeve 199 is accommodated within a retaining clip 243. The retaining clip 243 has a clamping lug 251 which is passed through a hole 253 in a clamping wall 255, wherein the clamping lug 251 presses against the clamping surface 138 within the opening 236 in the fastened state (see FIGS. 8 and 9). Accordingly, during assembly, the retaining clip 243 is pushed through the hole 253 orthogonally to the longitudinal alignment of the drive shaft 137 until the clamping lug 251 is arranged in a clamped manner within the opening 236. Because the retaining clip 243 is located at a position of the drop-shaped steering wheel 233 with the largest radius, it has the same haptics when operated by means of the articulation lever 141 as a circular steering wheel with a consistently maximum radius, despite the otherwise reduced size of the drop-shaped steering wheel 233.

Thus, a handpiece 121 is provided with a modularly adaptable control apparatus 131, 231, in which both the steering wheel 133, 233 and a clip 143, 243 for securing the pull wires 195, 197 to the steering wheel 133, 233 are flexibly adaptable, so that similar, familiar haptics is ensured for the user for steering wheels 133, 233 with both small and large diameters.

### LIST OF REFERENCE SIGNS

101 Endoscope
103 Shaft
105 Flexible shaft portion
107 Bendable shaft portion
109 Camera head
111 Distal end
113 Proximal end
115 Proximal direction
117 Adapter
121 Handpiece
123 Housing
125 Upper handle shell
127 Lower handle shell
131 Control apparatus
133 Steering wheel
135 Guide groove
136 Recess
137 Drive shaft
138 Clamping surface
139 Hub
141 Articulation lever
143 Ring clip
145 Power cable
147 Valve body
151 First clamping element
152 Second clamping element
157 Guide element
159 Anti-rotation element
161 Securing clip
195 First pull wire
197 Second pull wire
199 Crimp sleeve
231 Control apparatus
233 Drop-shaped steering wheel
236 Opening
243 Retaining clip
251 Clamping lug
253 Hole
255 Clamping wall

1-18. (canceled)

19. A control apparatus for an endoscopic apparatus having a bendable shaft portion, the control apparatus comprising:
a control element;
an actuating element; and
at least one Bowden cable with at least one cable element, wherein the at least one cable element is guided at least partially along the control element and has a clamping device having at least one clamping element;
wherein the at least one clamping element is connected to the control element in a non-positive-locking and/or a positive-locking manner and the at least one cable element is fastened to the control element by the clamping device.

20. The control apparatus according to claim 19, wherein the control apparatus comprises at least a second Bowden cable having at least one cable element.

21. The control apparatus according to claim 19, wherein the control apparatus comprises a plurality of Bowden cables, each of the plurality of Bowden cables having at least one cable element.

22. The control apparatus according to claim 19, wherein the control element has at least one counter-clamping portion for engaging and/or enclosing by means of the at least one clamping element.

23. The control apparatus according to claim 22, wherein the counter-clamping portion is formed as a recess, an undercut and/or a shaped surface in and/or on the control element.

24. The control apparatus according to claim 19, wherein the control element is configured as a steering wheel having an axis of rotation.

25. The control apparatus according to claim 19, wherein the control element and/or the steering wheel is configured to be circular, substantially oval or drop-shaped.

26. The control apparatus according to claim 19, wherein the clamping device is configured to be at least partially ring-shaped.

27. The control apparatus according to claim 24, wherein the clamping device is fastened with the at least one clamping element in a fastening direction along the axis of rotation of the steering wheel.

28. The control apparatus according to claim 24, wherein the clamping device is fastened with the at least one clamping element in a fastening direction substantially orthogonal to the axis of rotation of the steering wheel.

29. The control apparatus according to claim 19, wherein the clamping device has at least two clamping elements, wherein the at least two clamping elements are disposed on opposite sides of the control element.

30. The control apparatus according to claim 19, wherein the control element has at least one groove configured to guide the at least one cable element at least partially along the control element.

31. The control apparatus according to claim 30, wherein the control apparatus has two cable elements, each cable element having a proximal end, wherein the proximal ends of the two cable elements are connected to one another by a connecting means.

32. The control apparatus according to claim 31, wherein the connecting means is disposed in the at least one groove of the control element and/or in the clamping device and is coupled to the control element by the clamping device.

33. The control apparatus according to claim 26, wherein the ring-shaped clamping device is disposed outside the groove of the control element and/or around the at least one cable element.

34. The control apparatus according to claim 19, wherein the clamping device includes at least one guide element for guiding the at least one cable element.

35. The control apparatus according to claim 19, wherein the clamping device includes at least one anti-rotation element configured to prevent rotation of the clamping device on the control element and/or the steering wheel.

36. A handpiece for a flexible endoscopic apparatus having a bendable shaft portion, wherein the handpiece comprises the control apparatus according to claim 1.

37. A flexible endoscopic apparatus comprising:
a shaft having a bendable shaft portion, wherein the bendable shaft portion can be bent by the at least one Bowden cable;
a handpiece arranged at a proximal end of the shaft;
the control apparatus according to claim 1.
